(19) United States
(12) Patent Application Publication
(10) Pub. No.: US 2024/0201185 A1
(43) Pub. Date: Jun. 20, 2024

(54) MARKERS OF AUTOIMMUNE DISEASES

(71) Applicants: SORBONNE UNIVERSITE, Paris (FR); INSTITUT NATIONAL DE LA SANTE ET DE LA RECHERCHE MEDICALE (INSERM), Paris (FR); ASSISTANCE PUBLIQUE – HOPITAUX DE PARIS, Paris (FR)

(72) Inventors: Cindy MARQUES, Le Plessis Robinson (FR); Paul REGNIER, Fontenay Sous Bois (FR); David SAADOUN, Neuilly Sur Seine (FR)

(73) Assignees: SORBONNE UNIVERSITE, Paris (FR); INSTITUT NATIONAL DE LA SANTE ET DE LA RECHERCHE MEDICALE (INSERM), Paris (FR); ASSISTANCE PUBLIQUE – HOPITAUX DE PARIS, Paris (FR)

(21) Appl. No.: 18/556,496

(22) PCT Filed: Apr. 21, 2022
(86) PCT No.: PCT/EP2022/060498

§ 371 Date: Oct. 20, 2023

(30) Foreign Application Priority Data
Apr. 21, 2021 (WO) PCT/IB2021/000307

(51) Int. Cl. G01N 33/564; G01N 33/569; G01N 33/574

(52) U.S. Cl. CPC G01N 33/564; G01N 33/56972; G01N 33/57407; G01N 2333/70553; G01N 2333/70578; G01N 2333/70596; G01N 2333/7158; G01N 2800/101

(57) ABSTRACT

The present invention is directed to methods of diagnosis and treatment of autoimmune, chronic inflammatory and lymphoproliferative diseases based on the identification of a population of pathogenic B and/or T cells showing a specific phenotype. These cells may be identified by their specific pattern of expression of marker proteins.

The present invention is directed to methods of diagnosis and treatment of autoimmune, chronic inflammatory and lymphoproliferative diseases based on the identification of several combinations of pathogenic B and/or T immune cells populations from blood which show specific combinations of given phenotypes. These cells may be identified by their specific pattern of both surface and intracellular protein markers for example by flow cytometry.

Here, the aforementioned diseases may include Sjögren's syndrome (SS) and its clinical subgroups, Rheumatoid Arthritis (RA), Systemic Lupus Erythematosus (SLE) and Cryoglobulinemia (Cryo).

Autoimmune diseases occur when the immune system of a vertebrate attacks the “Self” tissue rather than an infectious agent. Autoimmune disorders can furthermore be associated with the development of lymphoproliferative disorders such as lymphomas, which is of particular relevance for Primary Sjögren's syndrome (pSS).

pSS and SS (both acronyms are used indifferently in the present invention and designate the same condition) are a systemic autoimmune disease characterized by lymphocytic infiltration of the exocrine glands, in particular the salivary and lacrimal glands. The clinical manifestations are numerous and can involve almost all organs (kidneys, lungs, peripheral nerves etc.). T and B cells play a major role in the development of the disease. In particular, patients with SS have altered peripheral B-cell compartments, characterized by fewer circulating CD27+ memory B-cells potentially due to their abnormal differentiation into plasma cells (1). Antibody production is deregulated in most patients with secretion of antinuclear autoantibodies that target Ro/SSA or La/SSB antigen.

RA is a severe inflammatory disease that affects the joints which are gradually destroyed, resulting in a disability.

SLE is the most characteristic connective tissue disease. It primarily affects the skin and joints but can also be complicated by potentially severe visceral manifestations, particularly neurological or renal.

Finally, Cryo is a heterogeneous group of systemic, inflammatory and/or thrombotic diseases, defined by the presence in the patient's serum of specific immunoglobulins by their ability to precipitate in vitro at a temperature below 37° C. The presence of this cryoglobulinemia results in the partial or complete obliteration of medium or small caliber blood vessels at the origin of the clinical manifestations of the condition.

Inventors have recently shown impaired elimination of autoreactive B cells in SS patients, which may promote autoimmunity by increasing the likelihood of presentation of the autoantigen via major histocompatibility complex class II to T cells.

The threat to patients with SS is the development of lymphoma (10-16 times the risk of the general population). The existing clinico-biological tools of B activation (rheumatoid factor (RF), complement consumption, cryoglobulinemia, etc.) are insufficient to distinguish between the different hematological entities that are close to each other: pre-lymphoma, indolent lymphoma and aggressive lymphoma. It is therefore essential to shed light on new biomarkers that could guide clinicians in the clinical monitoring and diagnosis of aggressive lymphoma.

The dominant type of lymphoma in SS is marginal zone lymphoma (MZL) involving mainly extra nodal sites (i.e. salivary glands, parotids, etc.) but the origin of the cells and the mechanisms leading to their malignant transformation are poorly understood. MZL may progress to an aggressive diffuse large B-cell lymphoma. Prolonged survival of B cells and their excessive activity, probably related to increased production of B cell activating factor (BAFF), can lead to lymphoma. The non-random use of immunoglobulin (lg) variable domain genes (VH and VL) by SS-associated lymphoma B cells, and the demonstration that these lymphoma B cells can exert RF activity, support the hypothesis that lymphoma cells develop by a process involving the concept of self-antigen. Lymphoproliferation has been correlated with the presence of an unusual population of B cells with decreased expression of the complement receptor 2/CD21 in the disease.

Interestingly, this correlation with the presence of said unusual population of B and/or T cells can also be used to establish a diagnosis and/or monitor the evolution of other autoimmune diseases such as pSS/SS, Cryo, RA and SLE.

Nonetheless, over the past decade, it has become evident that T and B cells interaction and collaboration also underlies the development of many autoimmune responses. In particular, follicular helper T cells (Tfh) interact with B cells in the secondary lymphoid organs leading to B cells differentiation and maturation.

More precisely, this invention can be divided in two distinct axes:
1) The identification by flow cytometry in patient blood of pathogenic T and B cells subpopulations (each having their own set of markers) in a given combination that allows to estimate the risk of a SS patient to subsequently develop a lymphoma in the future;
2) The identification of pathogenic T and B cells subpopulations that, in given combinations, helps in the differential diagnosis of pSS/SS, RA, Cryo and SLE.

Despite the B and T cells interaction and collaboration is established, it remains difficult to identify the correct set of markers on these cells which have an impact on autoimmune diseases appearance and evolution. It is also difficult to isolate cell populations or subgroups of these expressing markers of interest representing autoimmune diseases; the present invention thus proposes to remedy the aforementioned problems.

The data collection and bioinformatic methods we used to establish these results and claims are described hereafter in the “Bioinformatic analyses of flow cytometry data” (see example).

### SUMMARY OF THE INVENTION

The present invention relates to the following objects:

Item 1. Method of diagnosing an autoimmune disease or a lymphoproliferative form or a chronic inflammatory form of an autoimmune disease in a subject, comprising obtaining a test sample from the subject and detecting the presence of pathogenic B cells in the test sample, that express the marker proteins CD19, CD27, CD21, CD11c, CXCR5, and optionally at least one marker protein selected in the group consisting of Tbet, CD95, FCRL3, FCRL5, IgM, IgD, CD24, CD38 and/or G6 at a different level compared to a baseline level established from a healthy donor sample and wherein the presence of said pathogenic B cells in the sample, identifies the subject as having or likely to develop the autoimmune disease or a lymphoproliferative form or a chronic inflammatory form of an autoimmune disease.

Item 2. Method of item 1, wherein said disease is SS or a lymphoproliferative form of SS and the expression of marker proteins, for those detected, is as follows:

| | | Variation from healthy |
|---|---|---|
| Marker proteins | Level of expression | donors (%) |
| CD19 | High | about +50% |
| CD27 | Low | about −30% |
| CD21 | Low | about −15% |
| CD11c | High to very high | about +50% |
| CXCR5 | high | about +5% |

Item 3. Use of CD19, CD27, CD21, CD11c, CXCR5, and optionally at least one marker protein selected in the group consisting of Tbet, CD95, FCRL3, FCRL5, IgM, IgD, CD24, CD38 and/or G6 as markers for the in vitro detection of pathogenic B cells.

Item 4. Method of detection of pathogenic B cells in a subject, comprising obtaining a biological sample from the subject and determining the level of cellular expression of CD19, CD27, CD21, CD11c, CXCR5, and optionally at least one marker protein selected in the group consisting of Tbet, CD95 and/or FCRL3, wherein expression CD19+CD27− CD21-CD11c++ CXCR5+ Tbet++CD95+ FCRL3+ for the markers for which the level cellular expression has been determined is indicative of pathogenic B cells.

Item 5. In vitro detection method of pathogenic B cells in a biological sample from a patient comprising the steps of:
(i) determining the level of cellular expression of CD19, CD27, CD21, CD11c, CXCR5, and optionally at least one marker protein selected in the group consisting of Tbet, CD95 and/or FCRL3 of B cells in the biological sample;
(ii) in vitro detection of the expression of CD19+CD27− CD21− CD11c++ CXCR5+ Tbet++CD95+ FCRL3+ in B cells from the biological sample, for the markers for which the level cellular expression has been determined, the detection of this expression denoting the presence of pathogenic B cells in the biological sample.

Item 6. Kit for the diagnosis of autoimmune disease or a lymphoproliferative form or a chronic inflammatory form of an autoimmune disease or for the detection of pathogenic B cells, comprising reagents, each being used to determine the expression level of one of the marker proteins C CD19, CD27, CD21, CD11c, CXCR5, and optionally Tbet, CD95, FCRL3, FCRL5, IgM, IgD, CD24, CD38 and/or G6 in a sample.

Item 7. Method to evaluate the efficacy of a treatment of an autoimmune disease or a lymphoproliferative form or a chronic inflammatory form of an autoimmune disease in a subject, comprising determining the expression of marker proteins CD19, CD27, CD21, CD11c, CXCR5, and optionally at least one marker protein selected in the group consisting of Tbet, CD95, FCRL3, FCRL5, IgM, IgD, CD24, CD38 and/or G6 in B cells in a sample taken from the subject before administering the treatment; detecting the presence of pathogenic B cells in a sample taken from the subject after administering the treatment; and comparing the level of expression of said marker proteins in the sample taken from the subject before administering the treatment to the level expression of said marker proteins in the sample taken from the subject after administering the treatment.

Item 8. Method of treating an autoimmune disease or a lymphoproliferative form or a chronic inflammatory form of an autoimmune disease in a subject comprising reducing the activity of pathogenic B cells present in the subject by administering to the subject at least one antibody or antibody fragment that specifically binds to a protein expressed by the pathogenic B cells.

Item 9. Method according to item 8 wherein said protein expressed by the pathogenic B cells are selected in the group consisting of CD19, CD27, CD21, CD11c, CXCR5, Tbet, CD95, FCRL3, FCRL5, IgM, IgD, CD24, CD38 and/or G6.

Item 10. Pharmaceutical composition comprising at least one antibody or antibody fragment that specifically binds to a protein expressed by the pathogenic B cells.

Item 11. The pharmaceutical composition according to item 10, wherein said protein is selected in the group consisting of CD19, CD27, CD21, CD11c, CXCR5.

Item 12. The pharmaceutical composition according to item 10, wherein said protein is selected in the group consisting of CD19, CD27, CD21, CD11c, CXCR5, Tbet, CD95 and/or FCRL3.

Item 13. The pharmaceutical composition according to item 10, wherein said protein is selected in the group consisting of CD19, CD27, CD21, CD11c, CXCR5, Tbet, CD95, FCRL3, FCRL5, IgM, IgD, CD24, CD38 and/or G6.

Item 14. A method for discriminating auto-immune diseases using a combination of markers from B cells and T cells said combination of markers allows to define a score used to make the distinction between different auto-immune diseases.

More particularly, the method of Item 14 is such that the B cells markers are selected from CD3, CD19, CD27, CD21, IgM, CD11c, CXCR5, Tbet, FcRL3, CD24, IgD, CD38, FCRL5 and CD95 and wherein T cells markers are selected from CD3, CD4, CXCR5, CD25, CD127, CCR6, CXCR3, ICOS, PD-1, FoxP3, IFNγ, TNFα, IL-17, IL-4 and IL-21.

Item 15. A method of diagnosing an autoimmune disease or a lymphoproliferative form or a chronic inflammatory form of an autoimmune disease in a subject, comprising obtaining a test sample from the subject and detecting the presence of pathogenic B and/or T cells in the test sample, that express the marker proteins selected amongst:

| | |
|---|---|
| Target: B cells | Target: T cells |
| CD3− CD19+ | CD3+ CD4+ |

| Mix B | Mix T | Mix Cytoks |
|---|---|---|
| CD3 | CD3 | CD3 |
| CD19 | CD4 | CD4 |
| CD27 | CXCR5 | CXCCR5 |
| CD21 | CD25 | CXCR3 |
| IgM | CD127 | CCR6 |
| CD11c | CCR6 | IL-21 |
| CXCR5 | CXCR3 | IL-4 |
| Tbet | ICOS | IL-17 |
| FcRL3 | PD-1 | IFNγ |
| CD24 | FoxP3 | TNFα |
| IgD | | |
| CD38 | | |
| FcRL5 | | |
| CD95 | | |

Item 16. A method of diagnosing an autoimmune disease among Sjögren's syndrome (SS), cryoglobulinemic vasculitis (Cryo), rheumatoid arthritis (RA), Systemic Lupus Erythematosus (SLE) in a subject, comprising obtaining a test sample from the subject and detecting the presence of pathogenic B and/or T cells in the test sample, that express specific phenotypes characterized by all or part of the above listed markers.

### DETAILED DESCRIPTION

The present invention relates to a method of diagnosing an autoimmune disease or a lymphoproliferative form or a chronic inflammatory form of an autoimmune disease in a subject, comprising obtaining a test sample from the subject and detecting the presence of pathogenic B cells in the test sample, that express the marker proteins CD19, CD27, CD21, CD11c, CXCR5 and optionally at least one marker protein selected in the group consisting of Tbet, CD95, FCRL3, FCRL5, IgM, IgD, CD24, CD38 and/or G6 at a different level compared to a baseline level established from at least one healthy donor sample and wherein the presence of said pathogenic B cells in the sample identifies the subject as having or likely to develop the autoimmune disease or a lymphoproliferative form or a chronic inflammatory form of an autoimmune disease.

According to other embodiments, the present invention also relates to a method of diagnosing an autoimmune disease or a lymphoproliferative form or a chronic inflammatory form of an autoimmune disease in a subject, comprising obtaining a test sample from the subject and detecting the presence of pathogenic B and/or T cells in the test sample, that express the marker proteins selected amongst:

| | |
|---|---|
| Target: B cells | Target: T cells |
| CD3− CD19+ | CD3+ CD4+ |

| Mix B | Mix T | Mix Cytoks |
|---|---|---|
| CD3 | CD3 | CD3 |
| CD19 | CD4 | CD4 |
| CD27 | CXCR5 | CXCCR5 |
| CD21 | CD25 | CXCR3 |
| IgM | CD127 | CCR6 |
| CD11c | CCR6 | IL-21 |
| CXCR5 | CXCR3 | IL-4 |
| Tbet | ICOS | IL-17 |
| FcRL3 | PD-1 | IFNγ |
| CD24 | FoxP3 | TNFα |
| IgD | | |
| CD38 | | |
| FcRL5 | | |
| CD95 | | |

Examples of autoimmune or chronic infectious diseases include, without limitation, systemic lupus erythematosus (SLE), Sjögren's syndrome (SS), cryoglobulinemic vasculitis (Cryo), rheumatoid arthritis (RA), common variable immunodeficiency and VIH and VHC chronic infections.

According to a first particular embodiment, the profile expression of the marker proteins from pathogenic B cells associated with the risk of developing a SS or a lymphoproliferative form of SS is as follows:

| | | Variation from healthy |
|---|---|---|
| Marker proteins | Level of expression | donors (%) |
| CD19 | High (also designated by +) | about +50% |
| CD27 | Low (also designated by −) | about −30% |
| CD21 | Low (also designated by −) | about −15% |
| CD11c | High to very high (also | about +50% |
| | designated by + or ++) | |
| CXCR5 | High (also designated by +) | about +5% |

Optional markers include Tbet, CD95, FCRL3, FCRL5, IgM, IgD, CD24, CD38, G6. G6 antibody is an anti-idiotypic monoclonal Ab that selectively binds to IGHV1-69 heavy chain germline gene 51p1 alleles that have been implicated in Ab responses and disease processes (DOI: 10.1016/j.celrep.2017.11.056).

By optional markers according to the present invention it is meant that the first five markers are mandatory, and the optional ones can be present alone or in any combination with the mandatory ones or other optional ones.

The profile expression of some of these optional the marker proteins associated with the risk of developing a SS or a lymphoproliferative form of SS is as follows:

| | | Variation from healthy |
|---|---|---|
| Marker proteins | Level of expression | donors (%) |
| Tbet | High to very high (also | about +3% |
| | designated by + or ++) | |
| CD95 | High (also designated by +) | about −15% |
| FCRL3 | High (also designated by +) | about −40% |

According to several specific embodiment, the set of marker proteins is composed of:
CD19, CD27, CD21, CD11c, CXCR5 and CD95;
CD19, CD27, CD21, CD11c, CXCR5 and FcrL3;
CD19, CD27, CD21, CD11c, CXCR5 and Tbet;
CD19, CD27, CD21, CD11c, CXCR5, CD95 and FcrL3;
CD19, CD27, CD21, CD11c, CXCR5, CD95 and Tbet;
CD19, CD27, CD21, CD11c, CXCR5, FcrL3 and Tbet;
CD19, CD27, CD21, CD11c, CXCR5, CD95, FcrL3 and Tbet.

The term subject refers to any animal subject, and particularly, any vertebrate mammals, including, but not limited to, primates, rodents, livestock and domestic pets. Preferred mammals for the method of the present invention include humans.

The term sample refers to any biological sample obtained from the subject that contains peripheral blood cells. The sample may be a biological fluid sample, such as blood. The sample may also be a tissue sample obtained from a lymph node, spleen biopsy, parotid or minor salivary glands.

A healthy donor is an individual for whom no disease has been diagnosed.

Preferably, the baseline level established from at least one healthy donor sample is established with at least 10 healthy donor samples.

Pathogenic B cells are a population of B cells harboring specific phenotypes and that are associated with.

Pathogenic T cells are a population of T cells harboring specific phenotypes and that are associated with.

Expression of marker proteins may be assessed using any known methods in the art. The term expression refers to protein translation or mRNA transcription.

Methods suitable for the detection of protein include any suitable method for detecting and/or measuring proteins from a cell or cell extract. Such methods include, but are not limited to, staining and/or sorting using flow cytometry, polymerase chain reaction, immunoblot (e.g., Western blot), enzyme-linked immunosorbant assay (ELISA), radioimmunoassay (RIA), immunoprecipitation, immunohistochemistry and immunofluorescence. Particularly preferred methods for detection of proteins include any single cell assay, including staining and/or sorting using flow cytometry, immunohistochemistry and immunofluorescence assays. Such methods are well known in the art. Furthermore, antibodies against cell surface or intracellular proteins described herein are known in the art and are described in the public literature, and methods for production of antibodies that can be developed against these proteins are also well known in the art.

Methods suitable for detecting mRNA include any suitable method for detecting and/or measuring mRNA levels from a cell or cell extract. Such methods include, but are not limited to: polymerase chain reaction (PCR), reverse transcriptase PCR (RT-PCR), in situ hybridization, Northern blot, sequence analysis, gene microarray analysis (gene chip analysis), RNA sequencing and detection of a reporter gene. Such methods for detection of transcription levels are well known in the art, and many of such methods are described in detail https://www.elsevier.com/books/rna-methodologies/farrell-jr/978-0-12-804678-4.

In a preferred embodiment, the presence of pathogenic B and/or T cells is determined by co-immunostaining or co-immunolabeling the cells in the sample with antibodies or antibody fragments that specifically recognize the marker proteins.

The method may include the step of determining the frequency of the cells (percentage or the total number) that express part or all the cell surface or intracellular proteins. The frequency may be determined by any known method. Such methods may include flow cytometry or any laser-based revelation method.

In a preferred embodiment, the frequency of the cells is determined by flow cytometry. Flow cytometry is a technique used to detect and measure physical and chemical characteristics of a population of cells or particles. In this process, a sample containing cells or particles is suspended in a fluid and injected into the flow cytometer instrument. The sample is focused to ideally flow one cell at a time through a laser beam, where the scattered light is characteristic to the cells and their components. Furthermore, cells are often labeled with fluorescent markers, so that light is absorbed and then emitted in different bands of wavelengths. Tens of thousands of cells can be examined within minutes and the gathered data are then immediately processed by a computer. Flow cytometry is routinely used in basic research, clinical practice, and clinical trials.

The guidelines for the use of flow cytometry in immunology are summarized in the study of Cossarizza et al. (Eur J Immunol, 2019 https://onlinelibrary.wiley.com/doi/10.1002/eji.201970107). The analysis of data generated by flow cytometry remains subjective since it deals with labelling intensity and not with absolute positivity or negativity of a membrane or intracellular marker, as summarized in the following article: https://www.nature.com/articles/nri3158-c1. To be more precise, the measured fluorescence can be substantially altered because of a high number of possible elements, such as the experimental conditions (temperature, reagents dilutions/lot, quality of samples, number of cells, etc.) during the staining (batch effect) or the acquisition parameters (platform used, lasers power, number of cells per second, cells clogging, etc.). Of note, two operators will never obtain the exact same staining even if they follow the same protocol with the same reagents in the same facility, simply because of the randomness of cells that are stained. This is why it is inappropriate to define a fixed threshold for negativity or positivity for a given marker that will be considered as immutable. Rather, it is better to decide on such a threshold directly within the acquired data, each sample being its own negative and positive control at the same time Wang, L. and Hoffman, R. A. 2017. Standardization, calibration, and control in flow cytometry. Curr. Protoc. Cytom. 79:1.3.1-1.3.27. doi: 10.1002/cpcy.14).

In a preferred embodiment, data of the marker proteins expression are treated with the Uniform Manifold Approximation and Projection (UMAP) algorithm that is a non-linear dimensionality reduction method that allows to represent a set of points from a high dimensional space in a low dimensional space (typically two or three) in the same way as the t-SNE (t-distributed stochastic neighbor embedding) algorithm (see: https://umap-learn.readthedocs.io/en/latest/index.html). Concretely, raw flow cytometry data-containing files are given as input for a specifically written R code which includes opening, formatting, transformation, normalization and reshaping of raw data. Then, these transformed data are given as input for the UMAP algorithm (implemented in the uwot R package) with n=2 dimensions to retain. After UMAP analysis, reduced data are formatted as a table which is used to reconstruct flow cytometry files compatible with our standard flow cytometry analysis softwares (FlowJo from BD Biosciences and/or Kaluza from Beckman-Coulter). These softwares allow us to visualize data as point clouds with which we can interact with in order to analyze and finally export data like plots or percentages. According to another embodiment, the present invention relates to the use of CD19, CD27, CD21, CD11c, CXCR5, and optionally at least one marker protein selected in the group consisting of Tbet, CD95, FCRL3, FCRL5, IgM, IgD, CD24, CD38 and/or G6 as markers for the in vitro detection of pathogenic B cells.

The present invention also relates to a method of detection of pathogenic B cells in a subject, comprising obtaining a biological sample from the subject and determining the level of cellular expression of CD19, CD27, CD21, CD11c, CXCR5, and optionally at least one marker protein selected in the group consisting of Tbet, CD95, FCRL3, FCRL5, IgM, IgD, CD24, CD38 and/or G6.

In a particular embodiment, said method consists in determining the level of cellular expression of CD19, CD27, CD21, CD11c, CXCR5, and optionally at least one marker protein selected in the group consisting of Tbet, CD95, and/or FCRL3 and expression CD19+CD27-CD21− CD11c++ CXCR5+ Tbet++CD95+ FCRL3+ for the markers for which the level cellular expression has been determined is indicative of pathogenic B cells.

The present invention further relates to an in vitro detection method of pathogenic B cells in a biological sample from a patient comprising the steps of determining the level of cellular expression of CD19, CD27, CD21, CD11c, CXCR5, and optionally at least one marker protein selected in the group consisting of Tbet, CD95, FCRL3, FCRL5, IgM, IgD, CD24, CD38 and/or G6.

In a particular embodiment, said method comprising the steps of:
(i) determining the level of cellular expression of CD19, CD27, CD21, CD11c, CXCR5 and optionally at least one marker protein selected in the group consisting of Tbet, CD95 and/or FCRL3 of B cells in the biological sample;
(ii) in vitro detection of the expression CD19+CD27-CD21-CD11c++ CXCR5+ Tbet++CD95+ FCRL3+ in B cells from the biological sample, for the markers for which the level cellular expression has been determined, the detection of this expression denoting the presence of pathogenic B cells in the biological sample.

According to an embodiment of said in vitro method, the presence of pathogenic B cells in the biological sample indicates that the patient has a risk to develop a lymphoproliferative form of an autoimmune disease.

Included in the present invention are kits for the diagnosis of autoimmune disease or a lymphoproliferative form or a chronic inflammatory form of an autoimmune disease or for the detection of pathogenic B cells, comprising reagents, each being used to determine the expression level of one of the marker proteins CD19, CD27, CD21, CD11c, CXCR5, and optionally at least one marker protein selected in the group consisting of Tbet, CD95, FCRL3, FCRL5, IgM, IgD, CD24, CD38 and/or G6 in a sample.

Said reagent may be e.g., a probe that hybridizes under stringent hybridization conditions to a nucleic acid molecule encoding the marker proteins; RT-PCR primers for amplification of mRNA encoding the marker proteins or a fragment thereof; and/or an antibody, antigen-binding fragment thereof or other antigen-binding peptide that selectively binds to the marker proteins.

The reagents of the kit of the present invention can be conjugated to a detectable tag or detectable label. Such a tag can be any suitable tag which allows for detection of the reagents and includes, but is not limited to, any composition or label detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like), radiolabels (e.g., 3H, 1251, 35S, 14C, or 32P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads. In addition, the reagents of the kit can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly effecting the activity and/or ability of the detection means to detect the desired target molecule.

Exemplary organic solid supports include polymers such as polystyrene, nylon, phenol-formaldehyde resins, acrylic copolymers (e.g., polyacrylamide), stabilized intact whole cells, and stabilized crude whole cell/membrane homogenates.

Exemplary biopolymer supports include cellulose, polydextrans (e.g., Sephadex®), agarose, collagen and chitin.

Exemplary inorganic supports include glass beads (porous and nonporous), stainless steel, metal oxides (e.g., porous ceramics such as ZrO2, TiO2, Al203, and NiO) and sand. Other existing kits consist of dried, pre-formulated antibody panels for the detection of rare events, immune function analysis, research on the immune system and certain clinical applications, such as the DURAClone panel from Beckman Coulter.

According to another object, the present invention relates to a method to evaluate the efficacy of a treatment of an autoimmune disease or a lymphoproliferative form or a chronic inflammatory form of an autoimmune disease in a subject, comprising determining the expression of marker proteins CD19, CD27, CD21, CD11c, CXCR5, and optionally at least one marker protein selected in the group consisting of Tbet, CD95, FCRL3, FCRL5, IgM, IgD, CD24, CD38 and/or G6 in B cells in a sample taken from the subject before administering the treatment; detecting the presence of pathogenic B cells in a sample taken from the subject after administering the treatment; and comparing the level of expression of said marker proteins in the sample taken from the subject before administering the treatment to the level expression of said marker proteins in the sample taken from the subject after administering the treatment.

The decrease of the expression level of the marker proteins indicates that the treatment allows the decrease of the pathogenic B cells and that the treatment is efficient. The present invention also relates to a method of treating an autoimmune disease or a lymphoproliferative form or a chronic inflammatory form of an autoimmune disease in a subject comprising reducing the activity of pathogenic B cells present in the subject by administering to the subject antibodies or antibodies fragments that specifically binds to a protein expressed by the pathogenic B cells; in a specific embodiment, said protein is selected in the group consisting of CD19, CD27, CD21, CD11c, CXCR5, Tbet, CD95, FCRL3, FCRL5, IgM, IgD, CD24, CD38 and/or G6, preferably CD19, CD11c, CXCR5, Tbet, CD95 and/or FCRL3.

The present invention also relates to pharmaceutical composition comprising at least one antibody or antibody fragment that specifically binds to a protein expressed by the pathogenic B cells.

The pharmaceutical composition according to the present invention comprises at least one antibody or antibody fragment that specifically binds to a protein selected in the group consisting of CD19, CD27, CD21, CD11c, CXCR5; preferably CD19, CD11c, CXCR5.

The pharmaceutical composition according to the present invention comprises at least one antibody or antibody fragment that specifically binds to a protein selected in the group consisting of CD19, CD27, CD21, CD11c, CXCR5, Tbet, CD95 and/or FCRL3; preferably CD19, CD11c, CXCR5, Tbet, CD95 and/or FCRL3.

The pharmaceutical composition according to the present invention comprises at least one antibody or antibody fragment that specifically binds to a protein selected in the group consisting of CD19, CD27, CD21, CD11c, CXCR5, Tbet, CD95, FCRL3, FCRL5, IgM, IgD, CD24, CD38 and/or G6.

The present invention further relates to a method of diagnosing an autoimmune disease or a lymphoproliferative form or a chronic inflammatory form of an autoimmune disease in a subject, comprising obtaining a test sample from the subject and detecting the presence of pathogenic B and/or T cells in the test sample, that express the marker proteins selected amongst:

| | |
|---|---|
| Target: B cells | Target: T cells |
| CD3− CD19+ | CD3+ CD4+ |

| Mix B | Mix T | Mix Cytoks |
|---|---|---|
| CD3 | CD3 | CD3 |
| CD19 | CD4 | CD4 |
| CD27 | CXCR5 | CXCCR5 |
| CD21 | CD25 | CXCR3 |
| IgM | CD127 | CCR6 |
| CD11c | CCR6 | IL-21 |
| CXCR5 | CXCR3 | IL-4 |
| Tbet | ICOS | IL-17 |
| FcRL3 | PD-1 | IFNγ |
| CD24 | FoxP3 | TNFα |
| IgD | | |
| CD38 | | |
| FcRL5 | | |
| CD95 | | |

Examples of autoimmune or chronic infectious diseases include, without limitation, Sjögren's syndrome (SS), cryoglobulinemic vasculitis (Cryo), rheumatoid arthritis (RA), Systemic Lupus Erythematosus (SLE), common variable immunodeficiency and VIH and VHC chronic infections.

Said markers proteins may be identified by the previously described methods including methods involving antibodies, in such a case and for technical reasons related to IFNγ, TNFα, IL-17, IL-4 and IL-21 markers (present in the Mix Cytoks), these are to be stained in an antibody mix distinct from the Mix T, using a separate protocol.

Each of the three mixes helps to quantify given B or T cells subpopulations that allow to conduct the methods of the invention.

In a specific embodiment, the present invention relates to a method of diagnosing SS, L-SS and lymphoma in a subject, comprising obtaining a test sample from the subject and detecting the presence of pathogenic B and/or T cells in the test sample, that express the marker proteins selected amongst:
CD3− CD19+CD21− CD27− IgM+ CXCR5+CD11c− FcRL3− Tbet− in B cells; and/or
CD3+CD4+ TNFα+ CXCR3− CCR6− IL-21− CXCR5− IL-17− IL-4- and IFNγ+ or IFNγ− in T cells.

The method of the invention may be conducted and gives reliable results with markers of only one cell population (B cells or T cells).

According to another embodiment, the present invention relates to the use of:
CD3− CD19+CD21− CD27− IgM+ CXCR5+CD11c− FcRL3− Tbet− in B cells; and/or
CD3+CD4+ TNFα+ CXCR3− CCR6− IL-21− CXCR5− IL-17− IL-4- and IFNγ+ or IFNγ− in T cells;
   as markers for the in vitro detection of pathogenic B and/or T cells.

The present invention also relates to a method of detection of pathogenic B and/or T cells in a subject, comprising obtaining a biological sample from the subject and determining the level of cellular expression of:
CD3 CD19 CD21 CD27 IgM CXCR5 CD11c FcRL3 and Tbet in B cells; and/or
CD3 CD4 TNFα CXCR3 CCR6 IL-21 CXCR5 IL-17 IL-4 and IFNγ in T cells; In a particular embodiment, said method consists in determining the level of cellular expression of:
CD3 CD19 CD21 CD27 IgM CXCR5 CD11c FcRL3 Tbet in B cells; and/or
CD3 CD4 TNFα CXCR3 CCR6 IL-21 CXCR5 IL-17 IL-4 IFNγ in T cells;
   for the markers for which the level cellular expression has been determined is indicative of pathogenic B and/or cells.

The present invention further relates to an in vitro detection method of pathogenic B and/or T cells in a biological sample from a patient comprising the steps of determining the level of cellular expression of:
CD3 CD19 CD21 CD27 IgM CXCR5 CD11c FcRL3 Tbet in B cells; and/or
CD3 CD4 TNFα CXCR3 CCR6 IL-21 CXCR5 IL-17 IL-4 IFNγ in T cells.

In a particular embodiment, said method comprising the steps of:
(i) determining the level of cellular expression of:
   CD3 CD19 CD21 CD27 IgM CXCR5 CD11c FcRL3 Tbet in B cells; and/or
   CD3 CD4 TNFα CXCR3 CCR6 IL-21 CXCR5 IL-17 IL-4 IFNγ in T cells;
      in the biological sample;
(ii) in vitro detection of the expression:
   CD3− CD19+CD21− CD27− IgM+ CXCR5+CD11c− FcRL3− Tbet− in B cells; and/or
   CD3+CD4+ TNFα+ CXCR3− CCR6− IL-21− CXCR5− IL-17− IL-4- and IFNγ+ or IFNγ− in T cells;
      from the biological sample, for the markers for which the level cellular expression has been determined, the detection of this expression denoting the presence of pathogenic B and/or T cells in the biological sample.

According to an embodiment of said in vitro method, the presence of pathogenic B and/or T cells in the biological sample indicates that the patient has a risk to develop a lymphoproliferative form of an autoimmune disease.

This may be determined by calculating a diagnostic score taking into account:
the relative abundance of the phenotype CD3− CD19+CD21− CD27− IgM+ CXCR5+CD11c− FcRL3− Tbet− in B cells (called B1 in FIG. 4): if B1 is superior or equal to 2%, then the first score is 1 and if B1 is superior or equal to 5%, then the first score is 2;
the relative abundance of the phenotype CD3+CD4+ TNFα+ CXCR3− CCR6− IL− 21− CXCR5− IL-17− IL-4− and IFNγ+ or IFNγ− in T cells (called C1 in FIG. 4): if C1 is superior or equal to 4%, then the second score is 1 and if C1 is superior or equal to 8%, then the second score is 2; and
the first and second scores are summed up to give the diagnostic score.

If said diagnostic score is 0 then the subject is healthy; if it is 1 or 2, the subject is much more likely to have SS or L-SS and if the diagnostic score is 3 or 4, the subject has a significantly increased risk of having a lymphoma.

Included in the present invention are kits for the diagnosis of autoimmune disease or a lymphoproliferative form or a chronic inflammatory form of an autoimmune disease or for the detection of pathogenic B cells, comprising reagents, each being used to determine the expression level of one of the marker proteins:
CD3 CD19 CD21 CD27 IgM CXCR5 CD11c FcRL3 Tbet in B cells; and/or
CD3 CD4 TNFα CXCR3 CCR6 IL-21 CXCR5 IL-17 IL-4 IFNγ in T cells;
   in a sample.

Said reagent are as previously described.

According to another object, the present invention relates to a method to evaluate the efficacy of a treatment of an autoimmune disease or a lymphoproliferative form or a chronic inflammatory form of an autoimmune disease in a subject, comprising determining the expression of marker proteins:
CD3 CD19 CD21 CD27 IgM CXCR5 CD11c FcRL3 Tbet in B cells; and/or
CD3 CD4 TNFα CXCR3 CCR6 IL-21 CXCR5 IL-17 IL-4 IFNγ in T cells;
in a sample taken from the subject before administering the treatment; detecting the presence of pathogenic B and/or T cells in a sample taken from the subject after administering the treatment; and comparing the level of expression of said marker proteins in the sample taken from the subject before administering the treatment to the level expression of said marker proteins in the sample taken from the subject after administering the treatment.

A decrease of the relative abundance of the B1 and C1 phenotypes indicates an improvement of the subject's condition; indeed, the decrease of the abundance of these phenotypes indicates that the treatment allows the decrease of the pathogenic B and/or T cells and that the treatment is efficient.

The present invention also relates to a method of treating an autoimmune disease or a lymphoproliferative form or a chronic inflammatory form of an autoimmune disease in a subject comprising reducing the activity of pathogenic B and/or cells present in the subject by administering to the subject antibodies or antibodies fragments that specifically binds to a protein expressed by the pathogenic B and/or T cells; in a specific embodiment, said protein is selected in the group consisting of:
CD3− CD19+CD21− CD27− IgM+ CXCR5+CD11c− FcRL3− Tbet− in B cells; preferably CD19+ IgM+ CXCR5+ and/or
CD3+CD4+ TNFα+ CXCR3− CCR6− IL-21− CXCR5− IL-17− IL-4− IFNγ+ in T cells; preferably CD3+CD4+ TNFα+ IFNγ+.

The present invention also relates to pharmaceutical composition comprising at least one antibody or antibody fragment that specifically binds to a protein expressed by the pathogenic B and/or T cells.

The pharmaceutical composition according to the present invention comprises at least one antibody or antibody fragment that specifically binds to a protein selected in the group consisting of:
CD3, CD19, CD21, CD27, IgM, CXCR5, CD11c, FcRL3, Tbet in B cells; and/or
CD3, CD4, TNFα, CXCR3, CCR6, IL-21, CXCR5, IL-17, IL-4, IFNγ in T cells.

According to another embodiment, the present invention further relates to a method of diagnosing of an autoimmune disease among Sjögren's syndrome (SS), cryoglobulinemic vasculitis (Cryo), rheumatoid arthritis (RA), Systemic Lupus Erythematosus (SLE) in a subject, comprising obtaining a test sample from the subject and detecting the presence of pathogenic B and/or T cells in the test sample, that express the previously listed marker proteins.

Said method involves the determination of the several scores 1, 2.1, 2.2 and 3 representing the relative abundance of specific phenotypes B or T cells as detailed in FIG. 5D, wherein phenotypes T1 to T5, C1 to C4 and B1 to B3 are as detailed in FIG. 5B.

In this specific embodiment, the method of the invention comprises the determination of score 1;
(1) in case score 1 is 0 or 1, then score 2.1 is calculated;
   (1.1) in case score 2.1 is 0 or 1, then the subject is healthy;
   (1.2) in case score 2.1 is 2, 3 or 4, then the subject has SS;
(2) in case score 1 is 2, 3 or 4, then score 2.2 is calculated;
   (2.1) in case score 2.2 is 0, 1, 2 or 3, then score 3 is calculated;
      (2.1.1) in case score 3 is 0 or 1, then the subject has RA;
      (2.1.2) in case score 3 is 2 or 3, then the subject has SLE;
   (2.2) in case score 2.2 is 4, 5 or 6, then the subject has Cryo.

When calculating a score, if the considered phenotype is not is the indicated threshold, it counts for 0 otherwise it counts for 1.

Included in the present invention are kits for the diagnosis of autoimmune disease among Sjögren's syndrome (SS), cryoglobulinemic vasculitis (Cryo), rheumatoid arthritis (RA), Systemic Lupus Erythematosus (SLE), comprising reagents, each being used to determine the expression level of the marker proteins allowing the identification of phenotypes T1 to T5, C1 to C4 and B1 to B3 are as detailed in FIG. 5B.

The terms “antibody” and “antibodies” include polyclonal antibodies, monoclonal antibodies, humanized or chimeric antibodies, single chain Fv antibody fragments, Fab fragments, and F(ab′)2 fragments. Polyclonal antibodies are heterogeneous populations of antibody molecules that are specific for a particular antigen, while monoclonal antibodies are homogeneous populations of antibodies to a particular epitope contained within an antigen. Monoclonal antibodies and humanized antibodies are particularly useful in the present invention.

Antibody fragments that have specific binding affinity for a target of interest can be generated by known techniques.

The pharmaceutical composition of the invention is formulated to be compatible with its intended route of administration. Examples of routes of administration include parenteral, e.g., intrathecal, intra-arterial, intravenous, intradermal, subcutaneous, oral, transdermal (topical) and transmucosal administration.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; antibacterial agents such as benzyl alcohol or ethyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the injectable composition should be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a neuregulin) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

### FIGURES

FIG. 1. Expansion of a tissue like memory B cells (TLM) population in L-pSS patients. (A) Increased B cell population in L-pSS patients compared to pSS patients. (B) Increase in CD19/CD3 ratio in L-pSS patients compared to pSS patients. (C) Increased CD21− B cell count in L-pSS patients compared to pSS patients. (D, E) Distribution of the 4 B-cell subpopulations of interest, increase in the TLM level and decrease in the resting memory B cells (RM) level in L-pSS patients compared to pSS patients.

FIG. 2. UMAP analysis of B lymphocyte populations after flow cytometry. A) healthy donors, B) pSS patients and C) L-pSS patients.

FIG. 3. Bioinformatic workflow used for flow cytometry data processing and analysis. UML diagram showing the workflow we used to process flow cytometry raw data (FCS format) and analyze them.

FIG. 4. A given combination of CD4+ T cells and B lymphocytes subpopulations can be used to discriminate HD from SS/L-SS from Lymphoma.
(A) Heatmaps showing the absolute abundance of determined cell clusters (columns) in each medical condition of interest (row), either for Mix B (top), Mix Cytoks (bottom-left) and Mix T (bottom-right). Values are scaled column-wise. The color scale goes from black (lowest values) to white (highest values). Metaclusters defined as important and used in the later analyses are outboxed and labelled in white.
(B) Tables showing the actual phenotypes of the aforementioned B1 (top) and C1 (bottom) metaclusters individual components. “−” and “+” respectively denote negativity and positivity as compared to the global distribution of the cells.
(C) Histograms showing the actual B1 (left) and C1 (right) metaclusters abundances for each sample of each depicted medical condition.
(D) Truth table showing the scoring values and thresholds to apply to each sample of each medical condition of interest according to its measured abundance of B1 and C1 metaclusters.
(E) Histograms showing the final score computed for each patient of each medical condition when applying the method shown in (D) on metaclusters mentioned in (B). **: p-value<0.01, ***: p-value<0.001.

FIG. 5. A given combination of CD4+ T cells and B lymphocytes subpopulations can be used to construct successive but distinct scores which significantly discriminate HD from SS from Cryo from RA from SLE.
(A) Heatmaps showing the absolute abundance of determined cell clusters (columns) in each medical condition of interest (row), either for Mix B (top), Mix Cytoks (bottom-left) and Mix T (bottom-right). Values are scaled column-wise. The color scale goes from black (lowest values) to white (highest values). Metaclusters defined as important and used in the later analyses are outboxed and labelled in white.
(B) Tables showing the actual phenotypes of the aforementioned T1 to T5 (left), C1 to C4 (top-right) and B1 to B3 (bottom-right) metaclusters individual components. “−” and “+” respectively denote negativity and positivity as compared to the global distribution of the cells.
(C) Histograms showing the actual T1 to T5 (top), C1 to C4 (middle) and B1 to B3 (bottom) metaclusters abundances for each sample of each depicted medical condition.
(D) Truth tables showing for each score (Score 1, Score 2.1, Score 2.2 and Score 3) the scoring values and thresholds to apply to each sample according to its measured abundance of Tx, Cx and Bx metaclusters.
(E) Histograms showing the final score computed for each patient of each medical condition when applying the methods shown in (D) on metaclusters mentioned in (B).
(F) Decision tree showing the process to apply to any given sample in order to classify it as HD, SS, Cryo, RA or SLE. Thresholds for each score are indicated near their respective arrows. ***: p-value<0.001, ****: p-value<0.0001.

### EXAMPLES

### Materials & Methods

### Patients

Seventy-three patients with Sjögren syndrom (SS) treated in the Department of Internal Medicine and Clinical Immunology of Pitie-Salpêtrière Hospital (Paris, France) and 29 healthy donors were included. All SS patients were classified according to the 2016 American College of Rheumatology/European League Against Rheumatism classification criteria (ARD 2017-PMID: 27789466).

SS patients were classified in the “lymphoproliferation associated with SS (L-SS)” group if they had at least one of the following clinical or laboratory feature: salivary gland enlargement or peripheral lymphadenopathy, purpura, elevated rheumatoid factor, consumption of C4, hypergammaglobulinemia, cryoglobulinemia, monoclonal gammapathy or proven lymphoma.

The study was performed in accordance with the Declaration of Helsinki. All participants provided informed and written consents.

### Cell Isolation

Peripheral blood mononuclear cells (PBMCs) were obtained by Ficoll-separation from fresh whole blood samples.

### Flow Cytometry Analysis

PBMCs were stained for 30 min at room temperature with the following anti-human mouse monoclonal antibodies: Krome Orange (KO)- or Alexa Fluor 750 (AF750)-conjugated anti-CD3, Energy-Coupled Dye (ECD)-conjugated anti-CD19, Fluorescein Isothiocyanate (FITC)- or PE-Cyanine7 (PCy7)-conjugated anti-CD21, PE- or Allophycocyanin (APC)-conjugated anti-CD27, PCy7-conjugated anti-CD11c, APC-conjugated anti-CD95, FITC-conjugated anti-CD80, APC- or Pcy5.5-conjugated anti-CXCR5, PCy7- or AF750-conjugated anti-CD38, PerCP-Cy5.5 (PCy5.5)-conjugated anti-CD24, PE-conjugated anti-CD73, PE-conjugated anti-FCRL3 or anti-FCRL5, PE-conjugated anti-CD80, APC-conjugated anti-CD10, FITC-conjugated anti-IgM, PE-conjugated anti-IgD, Pacific Blue (PB)-conjugated anti-CD5, PerCP-conjugated anti-CD4, PCy7-conjugated anti-CD25, PE- or APC-conjugated anti-CCR6, Alexa Fluor 700 (AF700)-conjugated anti-CXCR3, Brilliant Violet 421 (BV421)-conjugated anti-CD127, KO-conjugated anti-PD-1, FITC-conjugated anti-ICOS and FITC-conjugated anti-IL1-R.

T-bet intracellular staining was performed using the PerFix-NC kit (Beckman Coulter) and pacific blue (PB)-conjugated anti-T-bet antibody according to the manufacturer's instructions.

FoxP3, IL-4, IL-17, IL-21, IFNγ and TNFα staining were performed using the Cytofix/Cytoperm buffer (BD PharMingen) and APC- or Alexa Fluor 647 (AF647)-conjugated anti-FoxP3, PE-conjugated anti-IL-4, Brilliant Violet 510 (BV510)-conjugated anti-IL-17, BV421-conjugated anti-IL-21, FITC-conjugated anti-IFNγ and PCy7-conjugated anti-TNFα antibodies following the same protocol as described above. The main difference for this last protocol is that cells were incubated in appropriated culture medium (RPMI supplemented with Penicillin/Streptomycin, L-glutamine and Bovine Serum Albumin) containing PMA and ionomycin for 4 h at 37° c. before performing the actual antibody staining as previously described.

Subsequent acquisition and analyses were performed with a Cytoflex flow cytometer platform and Kaluza analysis software, respectively (Beckman Coulter).

Processing and Analysis of Flow Cytometry Data with R

After their acquisition, raw flow cytometry files (FCS format) were opened with FlowJo version 10.8 in order to manually adjust the compensation matrix and to pre-process data. Lymphocyte-shaped events were extracted and doublets removed. Corresponding pre-processed data were then saved as new FCS files.

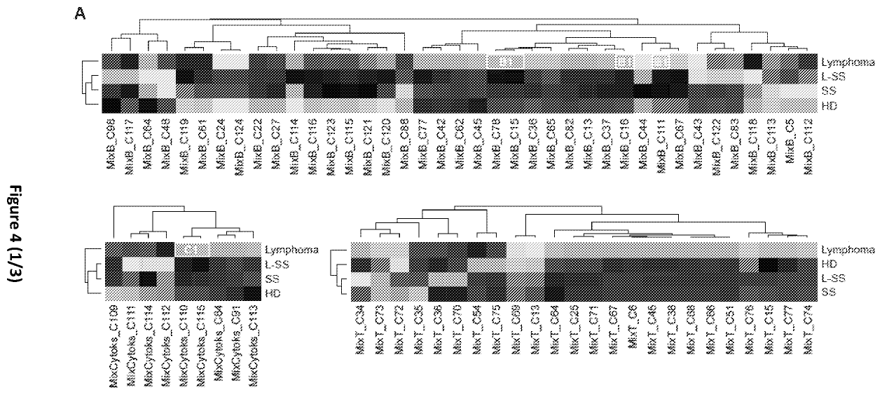

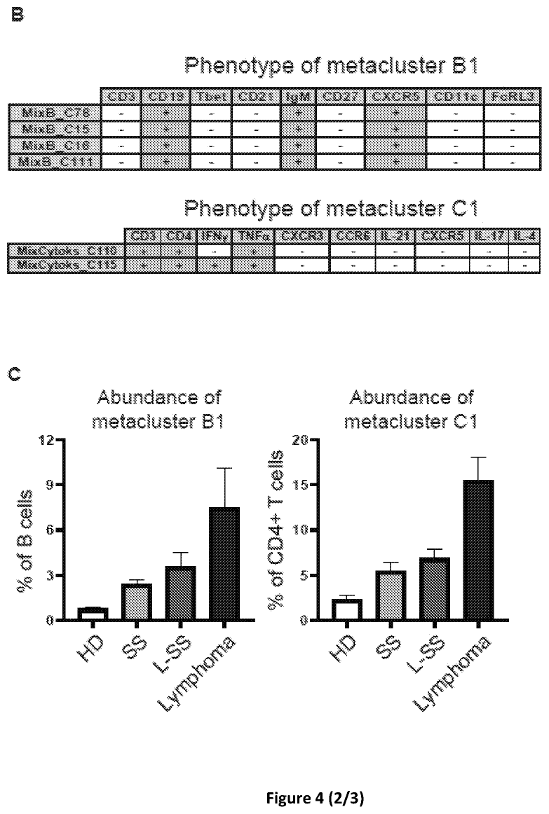

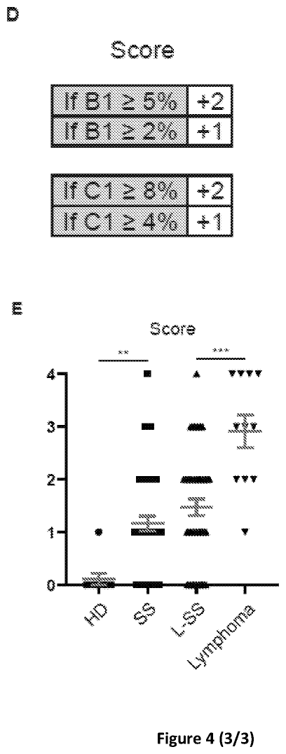

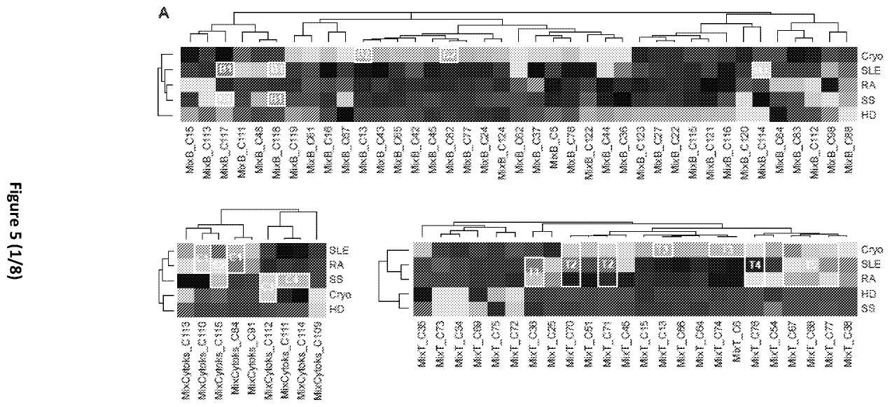

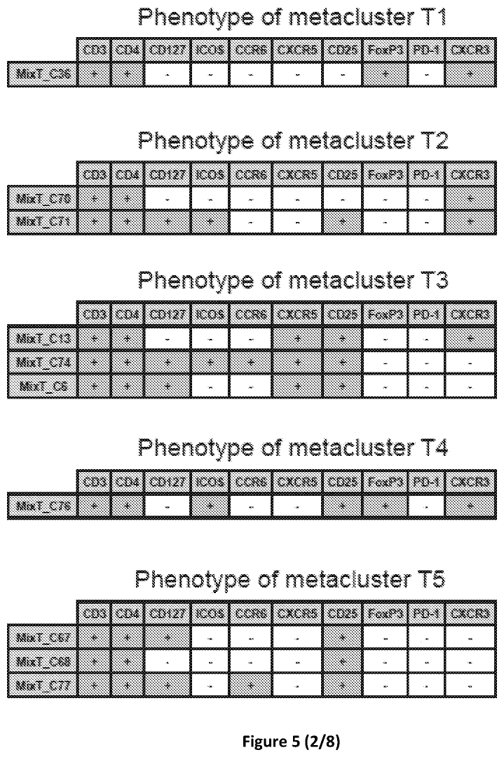

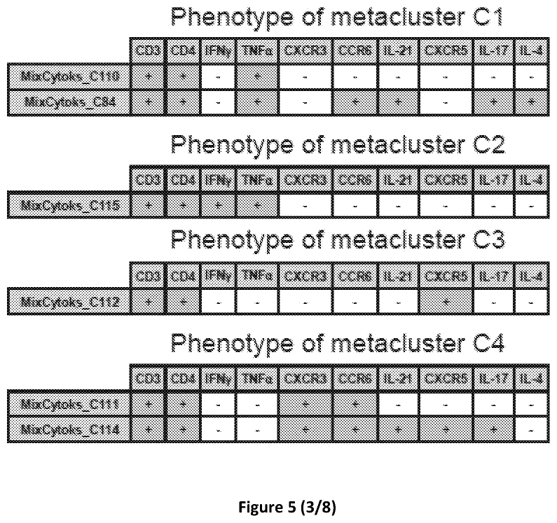

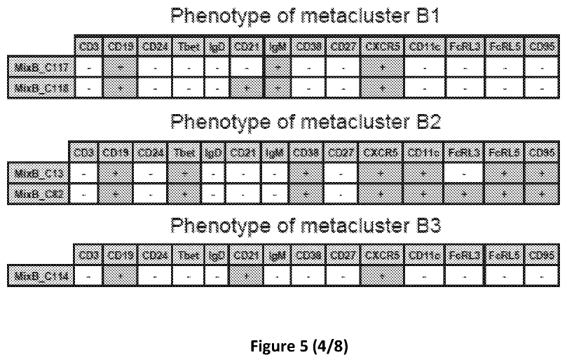

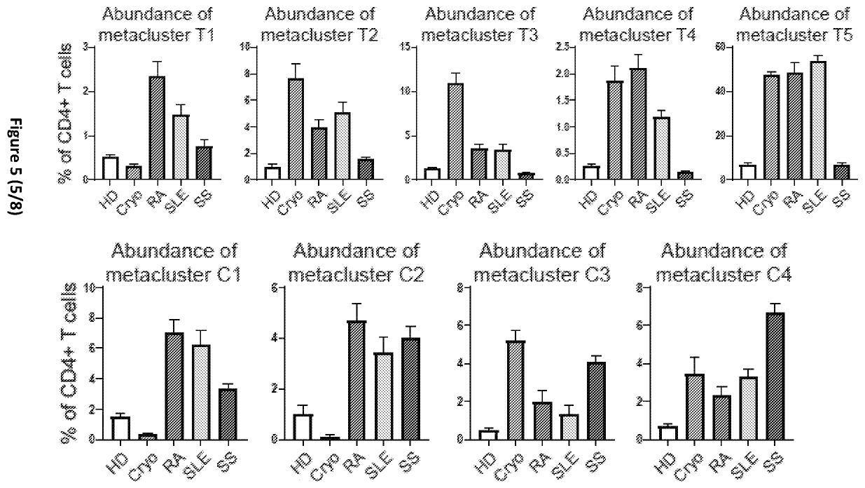

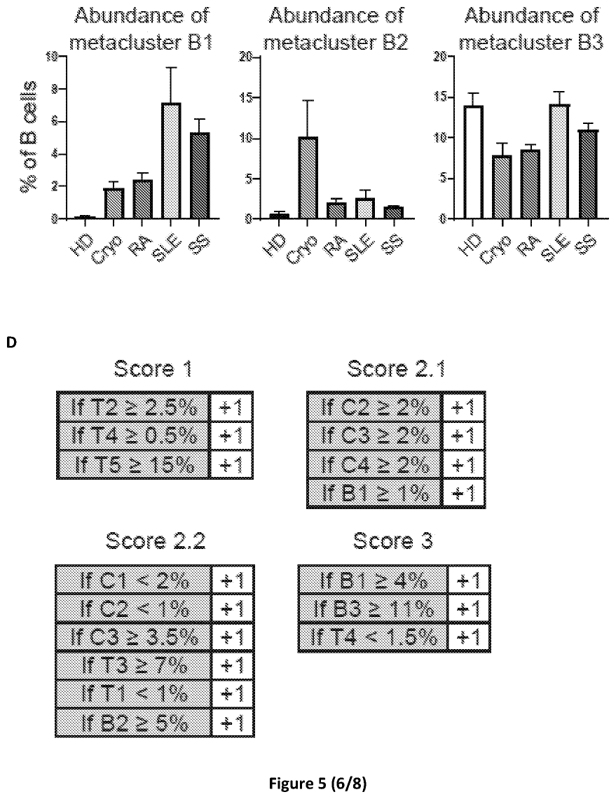

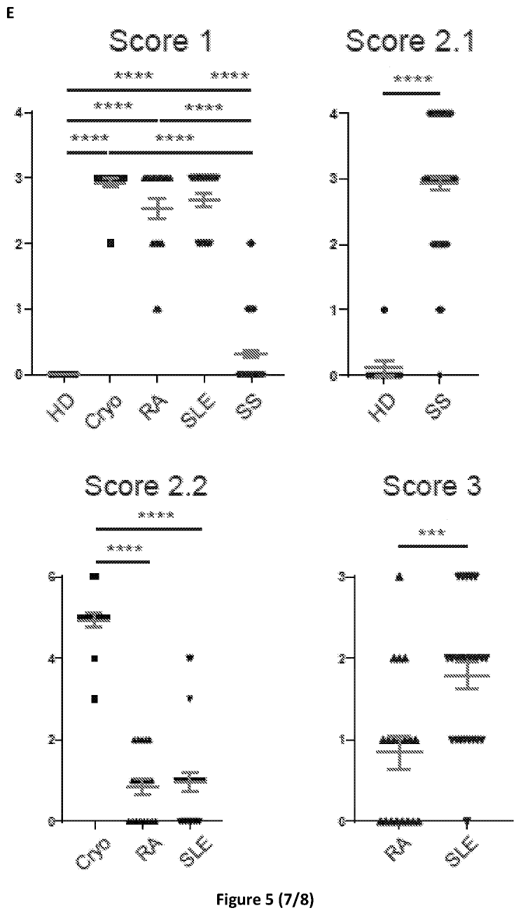

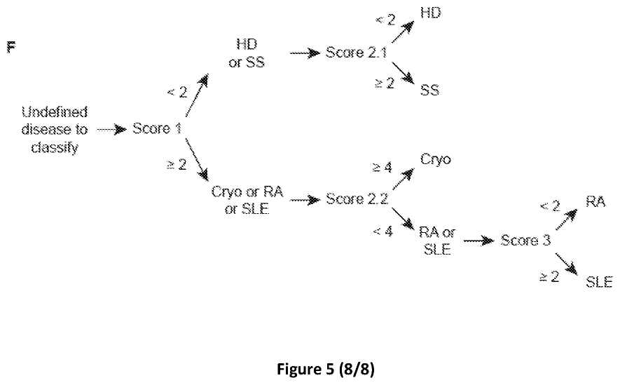

FIG. 3 summarizes the process used to analyze the collected data. Flow cytometry data (FCS format) from HD, SS, Cryo, RA and SLE samples were first imported in FlowJo version 10.8, where compensations were optimally adjusted and where lymphocyte-shaped single cells were exported to facilitate the subsequent bioinformatic analyses.

Then, newly exported FCS files were opened using flowCore and ggcyto Bioconductor R packages, according to their authors' instructions. Next, data were optimally logicle-transformed using again flowCore Bioconductor R package then normalized using gaussNorm method from flowStats Bioconductor R package (Hahne, F. et al. Per-channel basis normalization methods for flow cytometry data. Cytometry A 77, 121-131 (2010)). At this step, data contain all fluorescent parameters information for total lymphocytes and all samples. From here, two different lymphocytes populations were gated according to the antibody mix used: CD3− CD19+ cells (designated as “B cells”) for Mix B and CD3+CD4+ cells (designated as “CD4+ T cells”) for Mix T and Mix Cytoks, and corresponding data were therefore extracted for each sample.

Thereafter, these data were downsampled in order to make both medical conditions (HD, Cryo, SS, RA or SLE) and the number of samples inside each group contribute equally to the final dataset. Next, this downsampled dataset was analyzed by Uniform Manifold Approximation and Projection (UMAP) algorithm from uwot R package (Mclnnes, L., Healy, J. & Melville, J. UMAP: Uniform Manifold Approximation and Projection for Dimension Reduction. arXiv:1802.03426 [cs, stat] (2020)) and the subsequent UMAP model was extracted. Importantly, the included umap_transform method allowed us to add in the model the cells that were not originally retained in the downsampled dataset, in order to keep as many cells as possible in the future analysis process which helps to increase the number of studied cells as well as the global statistical power.

In parallel, hierarchical clustering of cells on the downsampled dataset has been performed in order to segregate these cells according to their phenotype and collapse the ones that share a similar phenotype together. This approach was intended to minimize as much as possible the number of identified cell clusters without altering the underlying biological messages within the datasets. Of note, it has been graphically checked that these determined clusters overlay in a logical manner on the UMAP projection topology, which is the sign of a non-artefactual cluster. Typical results of such analyses are presented in FIG. 4A and FIG. 5A.

At the end, the two UMAP dimensions, as well as transformed and normalized flow cytometry parameters, but also individual cells associations to their respective sample and group and their auto-attributed cluster were exported in separate files (one per antibody mix).

The final step of the workflow analysis was to keep the patients for whom the information about the three antibody mixes (which was not always the case) was available, and to analyze the remaining data using again UMAP, in order to globally visualize the patients repartition and segregation but also to eventually remove outliers in each patient groups. We ended up with nHD=9, nSLE=24, nCryo=15, nRA=19 and nSS=115 (decomposing as nSS=55, nL-SS=49 and nLymphoma=11). Once this was accomplished, heatmaps showing the abundances of each cluster within each medical condition of interest were computed (typically represented by the FIG. 4A and FIG. 5A), as well as the phenotype of each determined cluster (summarized in FIG. 4B and FIG. 5B). Using heatmaps representations makes it easier and more straightforward to determine which clusters are associated with a given disease or which are shared across several diseases. Then, these clusters percentages were eventually pooled (and referred as “metaclusters”) before assessing their true difference between the medical conditions of interest (typically represented in FIG. 4C and FIG. 5C). Finally, once a metacluster was found of interest, the phenotypes of the main cell populations composing it were extracted and summarized into tables (typically represented in FIG. 4B and FIG. 5B). Eventually, classification scores were established and applied to patients in order to more easily distinguish patients from clinical subgroups of interest (typically represented in FIG. 4D, FIG. 4E, FIG. 5D,

FIG. 5E and FIG. 5F).

### Results

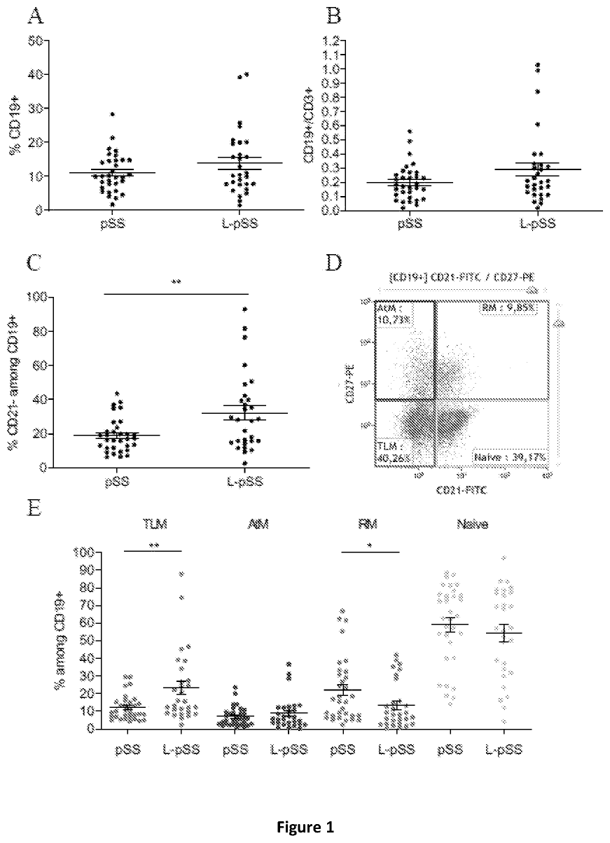

L-SS patients have a higher circulating B-cell count (FIG. 1A) and B-cell/T-cell ratio (FIG. 1B) than SS patients, although these results are not significant.

L-SS patients had a significantly higher CD21− B cell count than SS patients (32.14% vs. 18.99%, p=0.0043) (FIG. 1C).

Within the subpopulations of interest, L-SS patients had a higher TLM (23.27% vs. 12.01%, p=0.0044) and lower MR (13.53% vs. 21.96%, p=0.0473) than SS patients (FIGS. 1D and 1E).

To confirm these results in an unsupervised manner and refine the phenotype of the subpopulations of interest, the flow cytometry data were analyzed using an algorithmic technology called Uniform Manifold Approximation and Projection (UMAP).

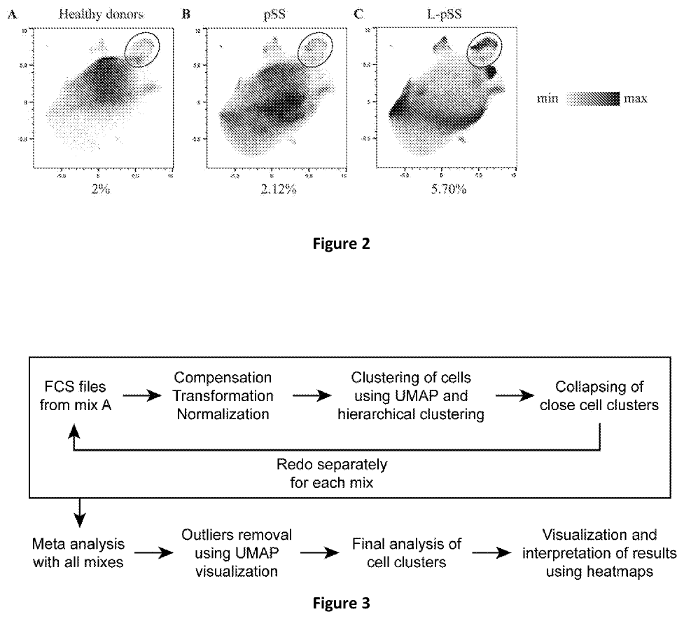

In FIG. 2, the B-cell compartment of all healthy donors (FIG. 2A), SS (FIG. 2B) and L-SS (FIG. 2C) patients are plotted together. The enrichment of a CD19+CD27− CD21-CD11c++ Tbet++ CXCR5+CD95+ FCRL3+ lymphocyte population is observed; representing respectively 2%, 2.12% and 5.70% of the B lymphocyte populations in these three groups. This method of analysis confirms in an unsupervised way the expansion of CD21-populations in L-SS patients in comparison with SS, and allows to refine the phenotype of these cells with the identification of surface and intracellular markers of interest.

Once the final biomarkers set is defined, each new subject sample will be processed the same way in order to determine the frequency of the pathogenic B cell population. A threshold fixing an abnormal abundance of such pathogenic B cell population will be defined and used to classify each new subject tested.

In a second set of assays aimed to detect markers of lymphoma risk within SS patients, the method described above leads to the heatmaps presented in the FIG. 4A which show all the identified cell clusters for each antibody mix in the 4 clinical groups of interest (HD, SS, L-SS and Lymphoma). From them, several groups of clusters were isolated (called metaclusters) that are progressively enriched through lymphoproliferation (B1 and C1, framed in white). From this information, the related phenotypes of these cell metaclusters has been extracted and organized them in a table (FIG. 4B). More precisely, metacluster B1 is composed of 4 distinct B subpopulations that all share the same phenotype (CD3-CD19+CD21− CD27− IgM+ CXCR5+CD11c− FcRL3− Tbet-), and metacluster C1 is composed of 2 distinct T cells subpopulations (CD3+CD4+ IFNγ+ TNFα+ CXCR3− CCR6− IL-21− CXCR5-IL-17− IL-4- and CD3+CD4+ IFNγ− TNFα+ CXCR3− CCR6− IL-21− CXCR5− IL-17− IL-4−). The FIG. 4C, generated using results presented in FIG. 4A, shows the absolute abundances of B1 and C1 metaclusters within B and T cells, respectively. This figure shows that metaclusters B1 and C1 allow to clearly distinguish HD versus SS/L-SS as well as SS/L-SS versus Lymphoma and accumulate through lymphoproliferation (from SS to Lymphoma). Furthermore, a score to classify HD, SS, L-SS and Lymphoma patients has been defined using the truth table presented in FIG. 4D. Applying this score to the entire patients set leads to the FIG. 4E, which shows that the computed score allows a clear distinction between HD versus SS/L-SS as well as SS/L-SS versus Lymphoma groups. To summarize, the method of the invention here involves the specified T and B cell metaclusters and involved subpopulations to measure (FIG. 4A and FIG. 4B) as well as their phenotype (FIG. 4C) and the associated classification score methodology (FIG. 4D and FIG. 4E).

For the purpose of diagnosis of different autoimmune diseases between them, the same method as previously mentioned was applied. The only changes here are reflected in the higher number of the metaclusters and cell populations used as well as the clinical groups of interest (here HD, SS, Cryo, RA and SLE). The obtained results are presented using heatmap representation in the FIG. 5A which allows to isolate metaclusters of B and T cells that can be used to best distinguish between the aforementioned diseases. In this case, 3 different B cell metaclusters (B1 to B3) (accounting for 5 different B cell populations) and 9 different T cell metaclusters (T1 to T5 and C1 to C4) accounting for 16 different T cells populations were defined and used to establish a diagnostic of Cryo, RA, SLE or SS versus HD. All these phenotypes are presented in tables shown in FIG. 3B. The FIG. 3C, generated using results presented in FIG. 5A, shows the absolute abundances of B1 to B3, T1 to T5 and C1 to C4 metaclusters within B and T cells, respectively. We also defined 4 scores (Score 1, Score 2.1, Score 2.2 and Score 3) based on the previously identified B and T cells metaclusters to better distinguish between autoimmune diseases. Each score contains a defined set of metaclusters to measure, as well as their associated thresholds to use for the scoring. The details of these scores are presented in FIG. 5D. Their application on the patients set leads to the classification shown in FIG. 5E. Importantly, what is an additional but crucial point for this claim is the decision tree shown in FIG. 5F. More precisely, these metaclusters and cell populations cannot be used “as is” to diagnose these 4 diseases, but rather should be used concomitantly with the decision tree, in order to progressively eliminate one or several diseases. In fact, each of the presented cell populations can be specifically associated with one or more autoimmune diseases. Furthermore, it is highly unlikely that a given cell population of T or B cells alone will define a specific disease by itself. This is why the combination of several markers and cell populations in several immune cells compartments (T and B cells here) is the most effective way to find combinations that are specifically associated with a given disease. The decision tree is based on progressive refining of the diagnosis using 4 different scores, each one representing a “step” closer towards a certain disease.
1. Glauzy, S et al. Defective Early B Cell Tolerance Checkpoints in Sjogren's Syndrome Patients. Arthritis & Rheumatology. 2017

1. A method of diagnosing of pSS, L-pSS and lymphoma in a subject, comprising obtaining a test sample from the subject and detecting the presence of pathogenic B cells in the test sample, that express the marker proteins CD19, CD27, CD21, CD11c, CXCR5, and optionally at least one marker protein selected in the group consisting of Tbet, CD95, FCRL3, FCRL5, IgM, IgD, CD24, CD38 and/or G6 at a different level compared to a baseline level established from a healthy donor sample and wherein the presence of said pathogenic B cells in the sample, identifies the subject as having or likely to develop the autoimmune disease or a lymphoproliferative form or a chronic inflammatory form of an autoimmune disease.

2. The method of claim 1, wherein the expression of marker proteins, for those detected, is as follows:

| | | Variation from |
|---|---|---|
| Marker proteins | Level of expression | healthy donors (%) |
| CD19 | High | about +50% |
| CD27 | Low | about −30% |
| CD21 | Low | about −15% |
| CD11c | High to very high | about +50% |
| CXCR5 | high | about +5% |

3. A method for an in vitro detection of pathogenic B cells, the method comprising: detecting CD19, CD27, CD21, CD11c, and CXCR5, and optionally at least one marker protein selected from the group consisting of: Tbet, CD95, FCRL3, FCRL5, IgM, IgD, CD24, CD38 and G6, as markers for the in vitro detection of pathogenic B cells.

4. A method of detection of pathogenic B cells in a subject, comprising obtaining a biological sample from the subject and determining the level of cellular expression of CD19, CD27, CD21, CD11c, CXCR5, and optionally at least one marker protein selected in the group consisting of Tbet, CD95 and/or FCRL3, wherein expression CD19+CD27− CD21− CD11c++ CXCR5+ Tbet++CD95+ FCRL3+ for the markers for which the level cellular expression has been determined is indicative of pathogenic B cells.

5. An in vitro detection method of pathogenic B cells in a biological sample from a patient comprising the steps of:
(i) determining the level of cellular expression of CD19, CD27, CD21, CD11c, CXCR5, and optionally at least one marker protein selected in the group consisting of Tbet, CD95 and/or FCRL3 of B cells in the biological sample;
(ii) in vitro detection of the expression CD19+CD27− CD21− CD11c++ CXCR5+ Tbet++CD95+ FCRL3+ in B cells from the biological sample, for the markers for which the level cellular expression has been determined, the detection of this expression denoting the presence of pathogenic B cells in the biological sample.

6. A kit for the diagnosis of pSS, L-pSS and lymphoma in a subject or for the detection of pathogenic B cells, comprising reagents, each being used to determine the expression level of one of the marker proteins CD19, CD27, CD21, CD11c, CXCR5, and optionally Tbet, CD95, FCRL3, FCRL5, IgM, IgD, CD24, CD38 and/or G6 in a sample.

7. A method to evaluate the efficacy of a treatment of an autoimmune disease or a lymphoproliferative form or a chronic inflammatory form of an autoimmune disease in a subject, comprising determining the expression of marker proteins CD19, CD27, CD21, CD11c, CXCR5, and optionally at least one marker protein selected in the group consisting of Tbet, CD95, FCRL3, FCRL5, IgM, IgD, CD24, CD38 and/or G6 in B cells in a sample taken from the subject before administering the treatment; detecting the presence of pathogenic B cells in a sample taken from the subject after administering the treatment; and comparing the level of expression of said marker proteins in the sample taken from the subject before administering the treatment to the level expression of said marker proteins in the sample taken from the subject after administering the treatment.

8-16. (canceled)

17. The method of claim 1, comprising obtaining a test sample from said subject and detecting the presence of pathogenic T cells in the test sample, that express the marker proteins selected amongst: CD3+CD4+ TNFα+ CXCR3− CCR6− IL-21− CXCR5− IL-17− IL-4− IFNγ+ or − in T cells.

18. The kit according to claim 7, comprising additional reagents, each being used to determine the expression level of one of the marker proteins: CD3 CD4 TNFα CXCR3 CCR6 IL-21 CXCR5 IL-17 IL-4 IFNγ in T cells; in a sample.
